(19) United States
(12) Patent Application Publication
(10) Pub. No.: US 2016/0366316 A1
(43) Pub. Date: Dec. 15, 2016

(54) SKIN ANALYSIS DEVICE AND IMAGE CAPTURING MODULE THEREOF

(71) Applicant: HTC Corporation, Taoyuan City (TW)

(72) Inventor: Chung-Jung CHEN, Taoyuan City (TW)

(73) Assignee: HTC CORPORATION, Taoyuan City (TW)

(21) Appl. No.: 14/738,026

(22) Filed: Jun. 12, 2015

(51) Int. Cl. H04N 5/225; A61B 5/00; F21V 5/00; H04N 5/357; F21V 8/00

(52) U.S. Cl. CPC H04N 5/2256; H04N 5/2254; H04N 5/3572; G02B 6/0046; G02B 6/0055; F21V 5/002; G02B 6/0051; G02B 6/0053; A61B 5/441; A61B 5/0077

(57) ABSTRACT

An image capturing module of a skin analysis device is provided, including a housing, a transparent element, an image sensing unit, an annular substrate, a plurality of light sources, and an annular optical mechanism. The transparent element is disposed at an end of the housing, and the image sensing unit is disposed in the housing and situated at a central axis of the image capturing module. The annular substrate is disposed in the housing, and the light sources are disposed on the substrate and surrounding the central axis. The light sources are situated between the transparent element and the image sensing unit in the direction of the central axis, and the annular optical mechanism is disposed in the housing and guides the light emitted from the light sources to the transparent element.

### BACKGROUND OF THE INVENTION

Field of the Invention

The application relates in general to an image capturing module, and in particular, to an image capturing module of a skin analysis device.

Description of the Related Art

Dermascope is a useful skin analysis tool which can capture skin images and provide easy and unambiguous analysis of the skin. Early detection of skin diseases, in particular skin cancer and melanoma, can be achieved by using the dermascope, so as to increase survival rates and reduce relative treatment costs.

Since traditional optical dermascopes capture skin images by reflecting light, the relative angles between the image sensing unit, the finger, and the light source have to be appropriately designed or adjusted for acquiring clear skin images. In general, flare patterns usually occur in the scanned skin images, and miniaturization of the dermascopes can be very difficult.

### BRIEF SUMMARY OF INVENTION

To address the deficiencies of conventional dermascopes, an embodiment of the invention provides an image capturing module of a skin analysis device, including a housing, a transparent element, an image sensing unit, an annular substrate, a plurality of light sources, and a plurality of light sources. The transparent element is disposed at an end of the housing, and the image sensing unit is disposed in the housing and situated at a central axis of the image capturing module. The annular substrate is disposed in the housing, and the light sources are disposed on the substrate and surrounding the central axis. The light sources are situated between the transparent element and the image sensing unit in a direction of the central axis, and the annular optical mechanism is disposed in the housing and guides the light emitted from the light sources to the transparent element.

### BRIEF DESCRIPTION OF DRAWINGS

The invention can be more fully understood by reading the subsequent detailed description and examples with references made to the accompanying drawings, wherein:

FIG. 1 is an exploded diagram of an image capturing module according to an embodiment of the invention;

FIG. 2 is a cross-section view of the image capturing module in FIG. 1 after assembly;

FIG. 3 is a cross-section view of an image capturing module according to another embodiment of the invention; and

FIG. 4 is an enlarged view that shows the light source and the optical mechanism in FIG. 3.

### DETAILED DESCRIPTION OF INVENTION

The making and using of the embodiments of a skin analysis device and an image capturing module thereof are discussed in detail below. It should be appreciated, however, that the embodiments provide many applicable inventive concepts that can be embodied in a wide variety of specific contexts. The specific embodiments discussed are merely illustrative of specific ways to make and use the embodiments, and do not limit the scope of the disclosure.

Unless defined otherwise, all technical and scientific terms used herein have the same meaning as commonly understood to one of ordinary skill in the art to which this invention belongs. It should be appreciated that each term, which is defined in a commonly used dictionary, should be interpreted as having a meaning conforming to the relative skills and the background or the context of the present disclosure, and should not be interpreted by an idealized or overly formal manner unless defined otherwise.

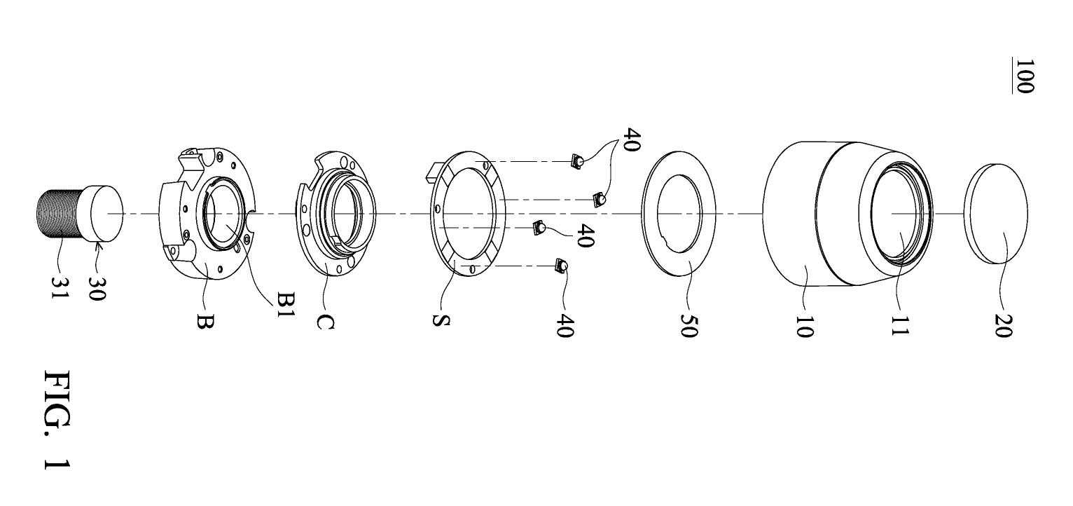

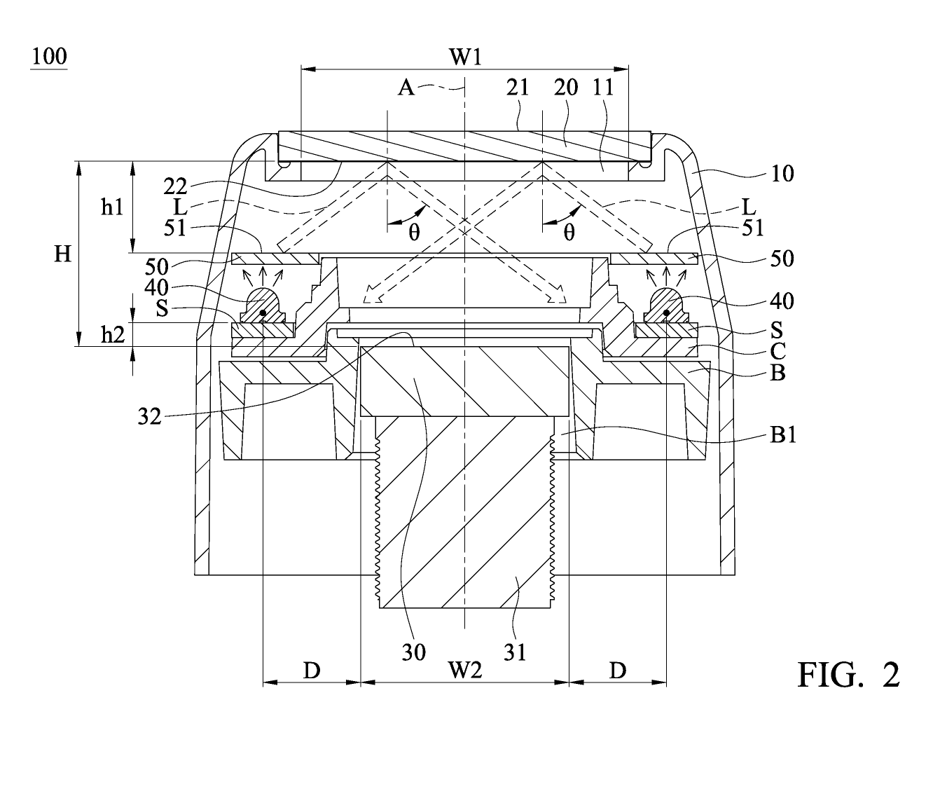

Referring to FIGS. 1 and 2, an image capturing module 100 according to an embodiment of the invention is included in a skin analysis device and electrically connected to a processing unit in the skin analysis device. Thus, the image capturing module 100 can capture skin images and transfer them to the processing unit for skin image analysis.

As shown in FIGS. 1 and 2, the image capturing module 100 primarily comprises a hollow housing 10, a transparent element 20, an image capturing unit 30, a plurality of light sources 40, an optical mechanism 50, a base B, a carrier C, and an annular substrate S. The image capturing unit 30 has a joining portion 31, and the image capturing module 100 can be screwed and secured into a side of the skin analysis device through the joining portion 31 with a threaded structure. When using the skin analysis device, the skin images can be acquired by the image capturing module for skin analysis.

In this embodiment, the transparent element 20 is disposed at the top of the housing 10 and adjacent to an opening 11 at the center of the housing 10. When using the skin analysis device, the skin of the user is in contact with a contact surface 21 of the transparent element 20, such that the image capturing unit 30 can capture images of the skin. Additionally, the image capturing unit 30, the light sources 40, the optical mechanism 50, the base B, the carrier C, and the substrate S are disposed in the housing 10. The image capturing unit 30 is disposed in a hole B1 at the center of the base B, and the carrier C is disposed on the substrate S for sustaining the light sources 40 and the substrate S.

In FIG. 2, the light sources 40 of this embodiment may be LEDs disposed on the substrate S and surrounding a central axis A of the image capturing module 100. The optical mechanism 50 may comprise optical lenses, such as diffusers or prisms, disposed above the light sources 40 to form an annular light emitting surface 51 above the light sources 40 and perpendicular to the central axis A. Thus, the incident angle θ of the light L when projected onto the transparent element 20 can be increased, so as to avoid small incident angle of light, and prevent flare effect due to direct light reflection by the lower surface 22 of the transparent element 20 to the sensing surface 32 of the image capturing unit 30.

Furthermore, to meet the requirements for both miniaturization of the image capturing module 100 and high opening ratio of the transparent element 20, the arrangement and relative positions of the aforementioned elements in the 3D space are designed in a specific manner. As shown in FIG. 2, the transparent element 20 and the image capturing unit 30 are spaced apart a distance H in the direction along the central axis A, the transparent element 20 and the optical mechanism 50 are spaced apart a distance h1 in the direction along the central axis A, and the image capturing unit 30 and the light sources 40 are spaced apart a distance h2 in the direction along the central axis A, wherein the light sources 40 are slightly higher than the image capturing unit 30. That is, the light sources 40 are located between the transparent element 20 and the optical mechanism 50 in the direction along the central axis A, wherein h2/H ranges from 0.15 to 0.2 (h2/H is 0.185 in this embodiment), and h1/H ranges from 0.4 to 0.6 (h1/H is 0.5 in this embodiment). Specifically, with the light sources 40 slightly higher than the image capturing unit 30, and the optical mechanism 50 above the light sources 40, a greater incident angle θ of the main light L projected from the light emitting surface 51 to the transparent element 20 can be achieved, so as to prevent a flare effect due to direct light reflection by the upper surface of the transparent element 20 to the image capturing unit 30.

As mentioned above, with appropriate arrangement of the transparent element 20, the image capturing unit 30, the light sources 40, and the optical mechanism 50, a greater incident angle θ of the main light L projected from the light emitting surface 51 to the transparent element 20 can be achieved to prevent the flare effect. In another aspect, the requirements of miniaturization of the image capturing module 100 and high opening ratio of the transparent element 20 can also be satisfied by the specific mechanism design. The transparent element 20 in this embodiment may be a transparent plate made by acrylic polymer or glass, wherein an opening 11 of the housing 10 is adjacent to the transparent element 20 and has a width W1, the image capturing unit 30 has a width W2, and W1/W2 ranges from about 0.8 to 1.2 (W1/W2 is 1 in this embodiment). Additionally, the center of the light source 40 and the image capturing unit 30 have a distance D in a horizontal direction, that is, the two light sources 40 on opposite sides have a distance of 2D+W2, wherein W1/(2D+W2) ranges from 0.5 to 0.8 ((W1/(2D+W2) is 0.59 in this embodiment). In other words, the invention not only prevents flare effect and achieves high opening ratio of the transparent element 20, the distance D between the image capturing unit 30 and the light source 40 can also be reduced to facilitate miniaturization of the image capturing module 100.

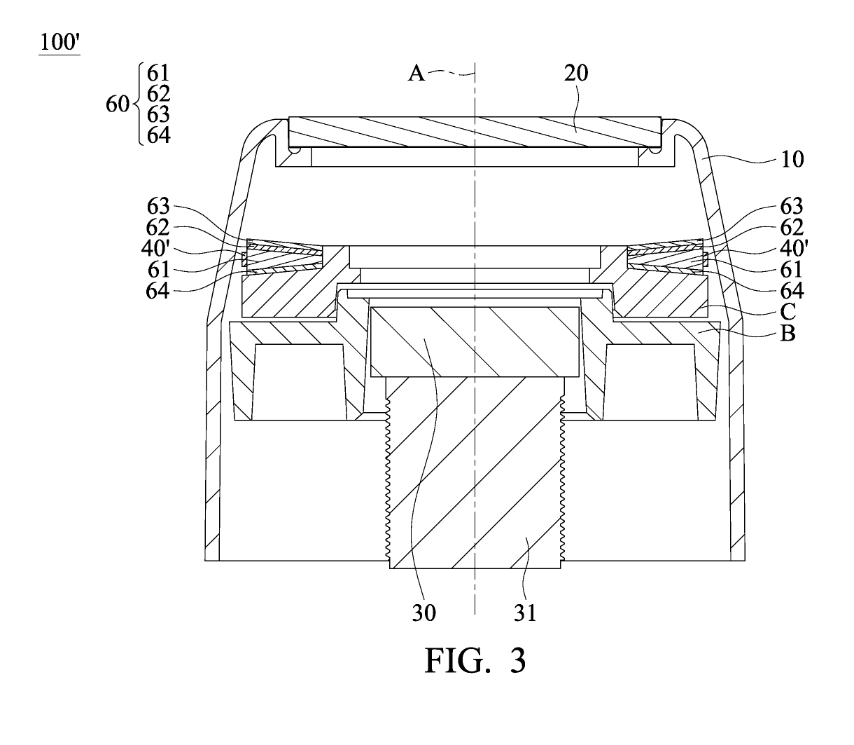

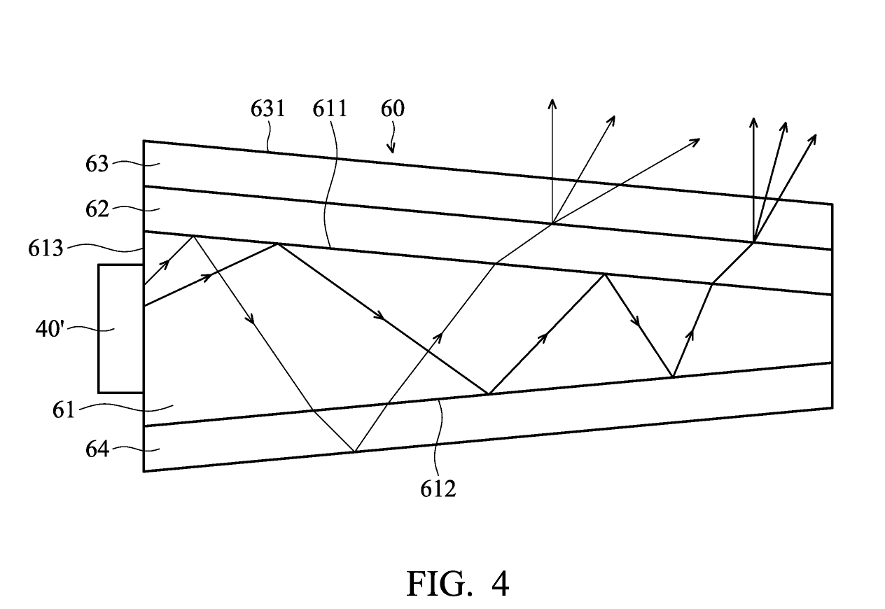

Referring to FIGS. 3 and 4, an image capturing module 100′ according to another embodiment of the invention comprises a plurality of light sources 40′ and an annular optical mechanism 60, respectively replacing the light sources 40 and the optical mechanism 50 in FIG. 2. It can be seen in FIG. 3, the optical mechanism 60 is disposed around the central axis A of the image capturing module 100′ and primarily includes a wedge-shaped light guide plate 61, a diffuser sheet 62, an optical film 63, and a reflecting sheet 64. The diffuser sheet 62 and the optical film 63 are disposed on a top side 611 of the light guide plate 61, and the reflecting sheet 64 is disposed on a bottom side 612 of the light guide plate 61. The diffuser sheet 62 is disposed between the light guide plate 61 and the diffuser sheet 62. As shown in FIGS. 3 and 4, a plurality of light sources 40′ are adjacent to a sidewall 613 of the light guide plate 61, wherein the sidewall 613 is between the top and bottom sides 611 and 612. When the light emitted from the light sources 40′ enters the light guide plate 61, it can be reflected by the reflecting sheet 64 and sequentially though the diffuser sheet 62 and the optical film 63. Finally, the light exits the optical mechanism 60 via a light emitting surface 631 at the top of the optical film 63. For example, the optical film 63 may comprise a microprism array or a microlens array, so as to control the light emitting angle and prevent the flare effect, wherein the light emitting surface 631 is angled with respect to the central axis A of the image capturing module 100′.

In summary, the invention provides an image capturing module with a compact size, which can prevent the flare effect and can be disposed in a skin analysis device for easy and unambiguous analysis of the skin (e.g. early detection of skin cancer and melanoma). Moreover, the skin analysis device can also be used as a fingerprint recognition device, wherein the image capturing module in the skin analysis device is electrically connected to a processing unit, so that a fingerprint image can be obtained by the image capturing module, and the fingerprint image can be transferred to the processing unit for fingerprint verification. For example, the skin analysis device (fingerprint recognition device) may connect to a portable electronic device, such as laptop computer, mobile phone, tablet computer, or any electronic equipment which requires a login or identification procedure, such as a personal computer, card reader, credit card swipe machine, Automated Teller Machine (ATM) for biometric authorization and identification, thereby facilitating transaction security of the system.

Although some embodiments of the present disclosure and their advantages have been described in detail, it should be understood that various changes, substitutions and alterations can be made herein without departing from the spirit and scope of the disclosure as defined by the appended claims. For example, it will be readily understood by those skilled in the art that many of the features, functions, processes, and materials described herein may be varied while remaining within the scope of the present disclosure. Moreover, the scope of the present application is not intended to be limited to the particular embodiments of the process, machine, manufacture, compositions of matter, means, methods and steps described in the specification. As one of ordinary skill in the art will readily appreciate from the disclosure of the present disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the appended claims should be accorded the broadest interpretation so as to encompass all such modifications and similar arrangements.

Use of ordinal terms such as “first”, “second”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

While the invention has been described by way of example and in terms of preferred embodiment, it is to be understood that the invention is not limited thereto. On the contrary, it is intended to cover various modifications and similar arrangements (as would be apparent to those skilled in the art). Therefore, the scope of the appended claims should be accorded the broadest interpretation to encompass all such modifications and similar arrangements.

What is claimed is:

1. An image capturing module, comprising:
a housing;
a transparent element, disposed at an end of the housing;
an image sensing unit, disposed in the housing and situated at a central axis of the image capturing module;
an annular substrate, disposed in the housing;
a plurality of light sources, disposed on the substrate and surrounding the central axis, wherein the light sources are situated between the transparent element and the image sensing unit in a direction of the central axis; and
an annular optical mechanism, disposed in the housing and guiding the light emitted from the light sources to the transparent element.

2. The image capturing module as claimed in claim 1, wherein the transparent element and the image sensing unit are spaced apart a distance H in the direction of the central axis, and the transparent element and the optical mechanism are spaced apart a distance h1 in the direction of the central axis, wherein the ratio of h1/H ranges from 0.15 to 0.2.

3. The image capturing module as claimed in claim 1, wherein the transparent element and the image sensing unit are spaced apart a distance H in the direction of the central axis, and the light sources and the image sensing unit are spaced apart a distance h2 in the direction of the central axis, wherein the ratio of h2/H ranges from 0.4 to 0.6.

4. The image capturing module as claimed in claim 1, wherein the housing has an opening, and the transparent element is adjacent to the opening, wherein the opening has a width W1, and the image sensing unit has a width W2, wherein the ratio of W1/W2 ranges from 0.8 to 1.2.

5. The image capturing module as claimed in claim 1, wherein the center of each of the light sources and the image sensing unit have a distance D therebetween in a horizontal direction, and the image sensing unit has a width W2 along the horizontal direction, wherein the ratio of W1/(2D+W2) ranges from 0.5 to 0.8.

6. The image capturing module as claimed in claim 1, wherein the image capturing module further comprises a base and a carrier, the image sensing unit is disposed in a hole of the base, and the carrier is disposed on the base to sustain the light sources and the substrate.

7. The image capturing module as claimed in claim 1, wherein the optical mechanism comprises a diffuser sheet or a prism sheet.

8. The image capturing module as claimed in claim 1, wherein the optical mechanism is disposed between the transparent element and the light sources.

9. The image capturing module as claimed in claim 1, wherein the transparent element comprises acrylic polymer or glass.

10. The image capturing module as claimed in claim 1, wherein the optical mechanism comprises a wedge-shaped light guide plate, an optical film, and a reflecting sheet, the optical film is disposed on a top side of the light guide plate, the reflecting sheet is disposed on a bottom side of the light guide plate, and the light sources are adjacent to a sidewall of the light guide plate, wherein the sidewall is between the top and bottom sides.

11. The image capturing module as claimed in claim 10, wherein the optical mechanism further comprises a diffuser sheet disposed on the top side of the light guide plate, between the light guide plate and the optical film.

12. The image capturing module as claimed in claim 10, wherein the optical film comprises a micro prism array or a micro lens array.

13. The image capturing module as claimed in claim 10, wherein the optical mechanism comprises a light emitting surface angled with respect to the central axis.

14. A skin analysis device, comprising the image capturing module as claimed in claim 1 and a processing unit, wherein the image capturing module is electrically connected to the processing unit.

15. The skin analysis device as claimed in claim 14, wherein the image sensing unit comprises a joining portion, and the image sensing unit is screwed and secured into a side of the skin analysis device via the joining portion.

16. The skin analysis device as claimed in claim 14, wherein the skin analysis device is connected to a laptop computer, mobile phone, tablet computer, personal computer, card reader, credit card swipe machine, or automatic teller machine.
